(19) United States
(12) Patent Application Publication
(10) Pub. No.: US 2013/0172869 A1
(43) Pub. Date: Jul. 4, 2013

(54) Systems and methods using sensors that resonate at a frequency equal to a resonance frequency of an ablated tissue

(71) Applicant: Jesse Bonfeld, (US)

(72) Inventor: Jesse Bonfeld, (US)

(21) Appl. No.: 12/362,261

(22) Filed: Jan. 29, 2009

Related U.S. Application Data
(63) Continuation-in-part of application No. 12/357,570, filed on Jan. 22, 2009, now Pat. No. 7,950,170.
(60) Provisional application No. 61/022,681, filed on Jan. 22, 2008.

(51) Int. Cl. A61B 18/18; A61N 1/39; A61N 1/37

(52) U.S. Cl. CPC A61B 18/18; A61N 1/3706; A61N 1/3937
USPC 606/33; 607/27; 607/5

(57) ABSTRACT

A method is provided of tissue ablation during a tissue ablation procedure. Ablation energy is applied by using a tissue ablation device to create an ablation at a tissue site. An ablation endpoint at the tissue site is detected by using an ablation endpoint device with one or more sensors that are positioned to monitor the ablation. The one or more sensors are selected from at least one of, a piezoelectric and a silicon MEMS sensor. Upon detecting the ablation endpoint, delivery of ablation energy to the tissue site ceases.

### CROSS-REFERENCE TO RELATED APPLICATIONS

This application claims the benefit of U.S. Ser. No. 12/357,570 filed Jan 22, 2009, which claims the benefit of U.S. Ser. No. 61/022,681, both of which applications are fully incorporated herein by reference.

### BACKGROUND

1. Field of the Invention

The present invention relates generally to detecting endpoints of ablation procedures, and more particularly to systems and methods that use one or more sensors that resonate at a frequency equal to the resonance frequency of the ablated tissue to determine endpoints of ablation procedures.

2. Related Art

During a medical ablation event, using electromagnetic energy including but not limited to RF, microwave and the like, the treating physician needs to know how far the ablation has proceeded in order to not over ablate.

Imaging methods have been used, without much success, to determine the endpoint of a medical ablation procedure.

In some medical applications, transvenous or intravenous ablation catheters with one or more electrodes are inserted into one or more heart cavities or put in contact with external areas of the heart to administer the ablation treatment to kill selected heart tissue. It is difficult to assess when to terminate the administration of the treatment in a manner that identifies when sufficient tissue has been destroyed to provide a clinically efficacious (transmural) linear ablation lesion. Particularly, “blind” or catheter-based ablation of cardiac tissue (such as to treat atrial fibrillation) can be more effective when patient-specific valid endpoints are used to recognize when a clinically efficacious lesion has been created. In the early ablation experience, acute termination followed by non-inducibility of the arrhythmia were used. Because these endpoints correlated poorly with long-term success, however, other parameters have been developed. Impedance and temperature measurements during the delivery of RF energy and the presence of conduction block after delivery of RF energy are the most common endpoints used in clinical practice.

Accordingly, there is a need for improved endpoint determinations during medical ablation procedures.

### SUMMARY

An object of the present invention is to provide improved endpoint determinations devices, and their uses for medical ablation procedures.

Another object of the present invention, acceleration or vibrations sensor devices are provided that are useful for determining the endpoints of medical ablation procedures.

These and other objects of the present invention are provided in a method of tissue ablation during a tissue ablation procedure. Ablation energy is applied by using a tissue ablation device to create an ablation at a tissue site. An ablation endpoint at the tissue site is detected by using an ablation endpoint device with one or more sensors that are positioned to monitor the ablation. The one or more sensors are selected from at least one of, a piezoelectric and a silicon MEMS sensor. Upon detecting the ablation endpoint, delivery of ablation energy to the tissue site ceases.

### BRIEF DESCRIPTION OF THE DRAWINGS

FIG. 1 is a cross sectional side view of a MEMS pressure sensor with selective encapsulation that can be used in one embodiment of the present invention.

FIG. 2 is a top view of the MEMS device of FIG. 1.

FIG. 3 is a cross sectional side view of a MEMS pressure sensor with selective encapsulation.

FIG. 4 is a top view of the MEMS device of FIG. 3.

FIG. 5 is a perspective view of a MEMS device in accordance with a third embodiment of a MEMS device that can be used with the present invention.

FIG. 6 is a cross sectional view of the MEMS device shown in FIG. 5.

FIG. 7 illustrates one embodiment of a tissue ablation device assembly with an ablation catheter and a sensor of the present invention.

FIGS. 8-10 illustrate one embodiment of a catheter based tissue ablation system with a sensor of the present invention.

### DETAILED DESCRIPTION

In one embodiment of the present invention an endpoint detection device includes one or more sensors. Suitable sensors include but are not limited to an acceleration or vibration sensor and the like. The sensor can be of either piezoelectric, pressure, thermal radiation, capacitance or silicon MEMS technologies to determine an endpoint of an ablation process and can be used for other non-ablation applications. Suitable piezoelectric transducers (sensors) are disclosed in U.S. Pat No. http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F4793194-h0#h0http://patft.uspto.gov/netacgi/nph-Parser?Sect2=PTO1&Sect2=HITOFF%p=1&u=%2Fnetahtml%2FPTO%2Fsearch-bool.html&r=1&f=G&1=50&d=PALL&RefSrch=yes&Query=PN%2F4793194-h2#h24,793, 194, http://patft.uspto.gov/netacgi/nph-Parser?Sect2=PTO1&Sect2=HITOFF&p=1&u=%2Fnetahtml.%2FPTO%2Fsearch-bool.html&r=1&f=G&1=50&d=PALL&RefSrch=yes&Query=PN%2F6988412-h0#h0http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F6988412-h2#h26,988,412, http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F7146865-h0#h0http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F7146865-h2#h27,146,865, U.S. Publication No. 2006/0117871 and U.S. Publication No. 2008/0084269. Suitable capacitance transducers (sensors) are disclosed in U.S. Patent No. http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F4999735-h0#h0http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F4999735-h2#h24,999,735. Suitable pressure transducers (sensors) are disclosed in U.S. Patent No. http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F4443293-h0#h0http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F4443293-h2#h24,443,293 and U.S. Publication No. 2002/0073783, Suitable thermal radiation sensors are disclosed in U.S. Patent No. http://patft.uspto.gov/netacgi/nph-Parser?Sect2=PTO1&Sect2=HITOFF&p=1&u=%2Fnetahtml%2FPTO%2Fsearch-bool.html&r=1&f=G&1=50&d=PALL&RefSrch=yes&Query=PN%2F6670538-h0#h0http://patft.uspto.gov/netacgi/nph-Parser?Sect2=PTO1&Sect2=HITOFF&p=1&=%2Fnetahtml%2FPTO%2Fsearch-bool.html&r=1&f=G&1=50&d=PALL&RefSrch=yes&Query=PN%2F6670538-h2#h26,670,538. All patents and publications recited in this paragraph being fully incorporated herein by reference.

The endpoint detection device can be used in a variety of therapeutic applications including but not limited to, activity monitoring for implantable defibrillators and pace makers, positioning in an ear canal to measure brain trauma, to monitor tissue ablation progress, provide diagnostic information and therapeutic treatment in a variety of applications including but not limited to neurology, and the like. In one embodiment the endpoint detection device can be used for long term implantable catheters.

In one embodiment, the endpoint detection device is used to monitor tissue ablation. The ablation can be performed using an ablation element which is typically electromagnetic. An ablation element within the intended meaning of the current invention may be adapted to ablate tissue in a variety of ways. For example, one suitable ablation element may be adapted to emit energy sufficient to ablate tissue when coupled to and energized by an energy source. Suitable examples of energy emitting ablation elements include without limitation: an electrode element adapted to couple to a direct current (DC) or alternating current (AC) source, such as a radiofrequency (RF) current source; an antenna element which is energized by a microwave energy source; a heating element, such as a metallic element which is energized by heat such as by convection or current flow, or a fiber optic element which is heated by light; a light emitting element, such as a fiber optic element which transmits light sufficient to ablate tissue when coupled to a light source; or an ultrasonic element such as an ultrasound crystal element which is adapted to emit ultrasonic sound waves sufficient to ablate tissue when coupled to a suitable excitation source as well as cryogenic sources and chemical ablation.

More detailed descriptions of radiofrequency (RF) ablation electrode designs which may be suitable in whole or in part as the ablating element according to the present invention are disclosed in U.S. Pat. No. 5,209,229 to Gilli; U.S. Pat. No. 5,487,385 to Avitall; and WO 96/10961 to Fleischman et al. More detailed descriptions of other energy emitting ablation elements which may be suitable according to the present invention are disclosed in U.S. Pat. No. 4,641,649 to Walinsky et al. (microwave ablation); and U.S. Pat. No. 5,156,157 to Valenta, Jr. et al. (laser ablation), all of which are incorporated in their entirety by reference thereto.

In addition, other elements for altering the nature of tissue may be suitable as “ablation elements” within the intended meaning of the current invention. For example, a cryoblation probe element adapted to sufficiently cool tissue to substantially alter the structure thereof may be suitable. Furthermore, a fluid delivery element, such as a discrete port or a plurality of ports which are fluidly coupled to a fluid delivery source, may be adapted to infuse an ablating fluid, such as a fluid containing alcohol, into the tissue adjacent to the port or ports to substantially alter the nature of that tissue. More detailed examples of cryoblation or fluid delivery elements such as those just described are disclosed in U.S. Pat. No. 5,147,355 to Friedman et al. and WO 95/19738 to Milder, respectively. The disclosures of these patents are incorporated in their entirety by reference thereto.

With the present invention, one or more sensors are provided that resonate at a frequency equal to the resonance frequency of the ablated tissue. This resonance frequency is different from the frequency of the non-ablated tissue. The sensor can be coupled to an external detection device. The external device indicates when the sensor is excited to its resonance frequency. The sensor can be coupled to the external detection device by cable, wireless and the like. Achieving a specific resonance frequency is used to determine the endpoint of the tissue ablation procedure.

As the ablation process proceeds tissue radiating from the ablation device is effected by the procedure. When the procedure reaches the tissue where the sensor or sensor array is positioned, the detector notifies the physician with an indication that the procedure should now be discontinued.

In one embodiment, the endpoint detection device has an array of sensors that are mounted to or encapsulated in a biocompatible material, or are manufactured from a biocompatible material. The array of sensors is positioned at an ablation site and can fully or partially surround the ablation site. The array of sensors is tuned to a specific resonance frequency of the ablated tissue. This resonance frequency is different from the resonance frequency of the non-ablated tissue. When the ablation has reached the sensor array and thus the desired endpoint, the array of sensors produces an electrical signal.

In one embodiment, the sensor is a piezoresistive sensor that has a substrate with two opposed surfaces. A dielectric insulative layer is on the a first surface of the substrate. A doped semiconductor layer is on top of the dielectric insulated layer. The semiconductor layer has a high resistivity. The doped semiconductor layer is annealed to one or more regions to lower resistivity of the semiconductor layer and define therein one or more sensor gauges of the annealed semiconductor material. Electrical contacts are adjacent to the annealed semiconductor material and overlay at least a portion of the annealed semiconductor material.

One embodiment of a suitable MEMS sensor that can be used with the present invention includes a housing and a sensor die that can be attached to the housing by an epoxy or silicone adhesive. Wire bonds provide an electrical connection between wire bond pads of the sensor die and a lead frame. A protective dam and an encapsulation gel material can be included as disclosed in U.S. Pat. No. 6,401,545, incorporated herein by reference.

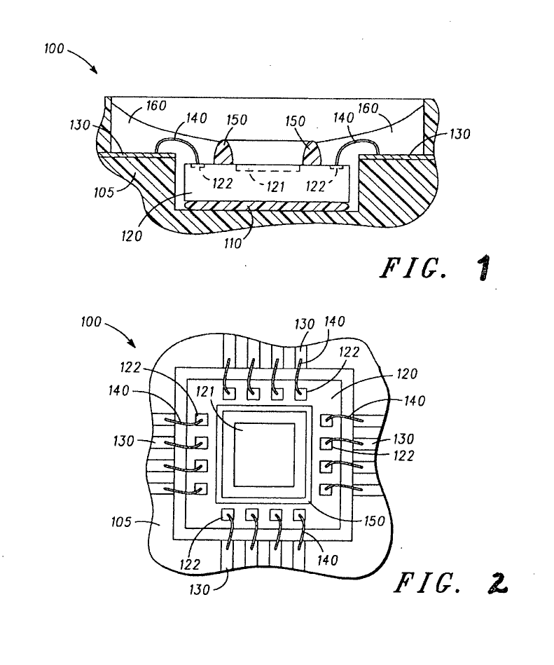

FIG. 1 is a cross-sectional side view of a MEMS sensor 100 with selective encapsulation that can be used in one embodiment of the present invention. MEMS pressure sensor 100 comprises a housing 105 (partially shown) which is typically made of a plastic material. A sensor die 120 is attached to plastic housing 105 by an epoxy or silicone adhesive 110. Wire bonds 140 provide an electrical connection between wire bond pads 122 of sensor die 120 and a lead frame 130. Also shown are a protective dam 150 and an encapsulation gel material 160, which serves as a protectant.

The particulars of the various elements, as well as the technique for fabricating the improved MEMS sensor 100, is as follows. The description of the various embodiments of the present invention is drawn primarily to a MEMS pressure sensor. However, the described embodiments of the present invention of selective encapsulation are applicable to a wide variety of MEMS sensors, including capacitive sensors which sense pressure, chemical, humidity, etc.

The common denominator of these types of MEMS sensors with regard to the various embodiments of the present invention, is a transducer element such as a capacitive diaphragm or membrane which is sensitive to some ambient condition and which, for optimal performance, should be free of encapsulation gel. However, the remainder of the package, other than the transducer element, should be encapsulated for environmental protection.

After a wafer containing numerous MEMS pressure sensor devices is diced into individual dies, each individual pressure sensor die 120 is attached to a housing 105, which is typically made of plastic, by conventional means. Typically an epoxy or silicone adhesive 110 is used to attach pressure sensor die 120 to the base of plastic housing 105.

Subsequent to attaching pressure sensor die 120 to plastic housing 105, wire bonds 140 are connected between die wire bond pads 122 and lead frame 130.

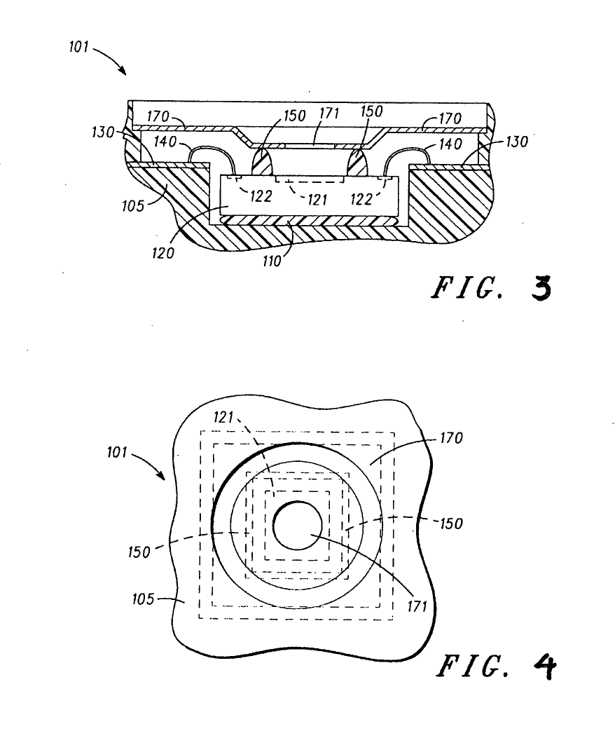

A subsequent step is to construct protective dam 150 between the outer perimeter of a pressure sensor diaphragm 121 and the inner perimeter formed by bond pads 122. Pressure sensor diaphragm 121 is typically located in the center portion of pressure sensor die 120 as shown in FIG. 3. Protective dam 150 is then cured at high temperature.

Following the curing of protective dam 150, MEMS pressure sensor 100 is ready for encapsulation. During this step an encapsulation gel 160 is dispensed into the wire bond cavity region that is located between protective dam 150 and plastic housing 105, thereby covering bond pads 122, portions of lead frame 130 and wire bonds 140. After selective encapsulation is completed, encapsulation gel 160 is cured.

By way of example, protective dam 150 is constructed of a fluorocarbon based material to achieve the best media compatibility, i.e., to protect the integrity of the wire bonds 140 from contamination by foreign matter. However, fluorocarbon type material is also typically the most costly. Other cost effective materials include silicone and fluorosilicone base materials. Typically similar materials are used for both protective dam 150 and encapsulation gel 160.

Protective dam 150 is typically constructed by forming it as a unit using a device such as a dispensing collet. A dispensing collet is a nozzle type device where the design of the output opening of the nozzle corresponds the design of protective dam 150. Thus, for rectangular shaped dams, the dispensing collet would have a rectangular shaped nozzle which would permit the formation of all four walls of the protective dam 150 simultaneously. The current design preferably uses a rectangular shaped protective dam 150 for consistency with the rectangular shaped pressure sensor diaphragm 121. However, other shape diaphragms and protective dams are contemplated including, but not limited to, circular configurations, triangular configuration, pentagonal configurations, or the like.

Alternatively, each of the four walls of protective dam 150 may be formed using a dispensing needle. In the dispensing needle method of protective dam construction, each of the dam walls is formed sequentially, as opposed to the dispensing collet method in which the dam walls are formed simultaneously. The dispensing needle essentially line draws each dam wall. Multiple passes for each wall can be made to control the height and width of protective dam 150.

The minimum height for protective dam 150 is preferably equal to approximately the loop height of wire bonds 140, i.e. the apogee of wire bonds 140 above pressure sensor die 120. The minimum height is driven by the requirement to insure complete encapsulation of wire˜bonds 140. The maximum height of protective dam 150 is that of plastic housing 105. However, in practice the height of protective dam 150 ranges between the apogee of wire bonds 140 and plastic housing 105 as shown in FIG. 2.

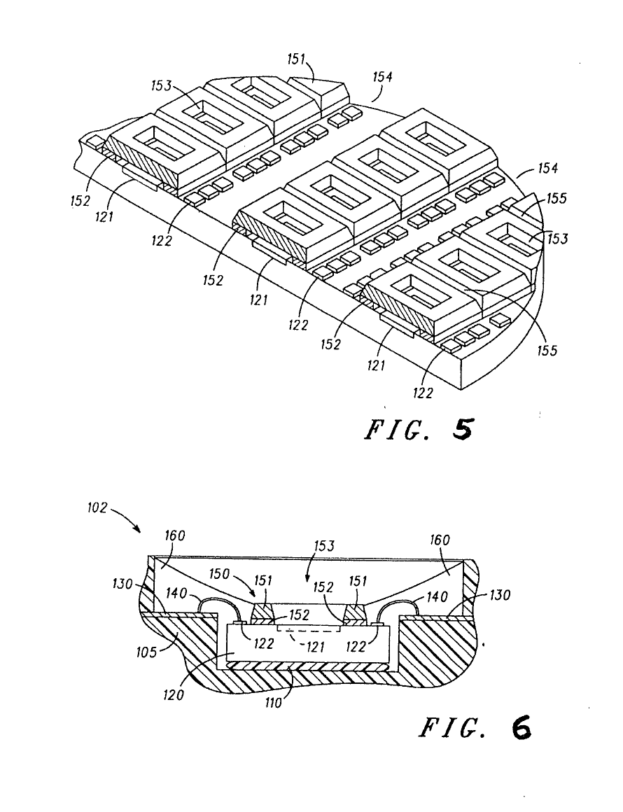

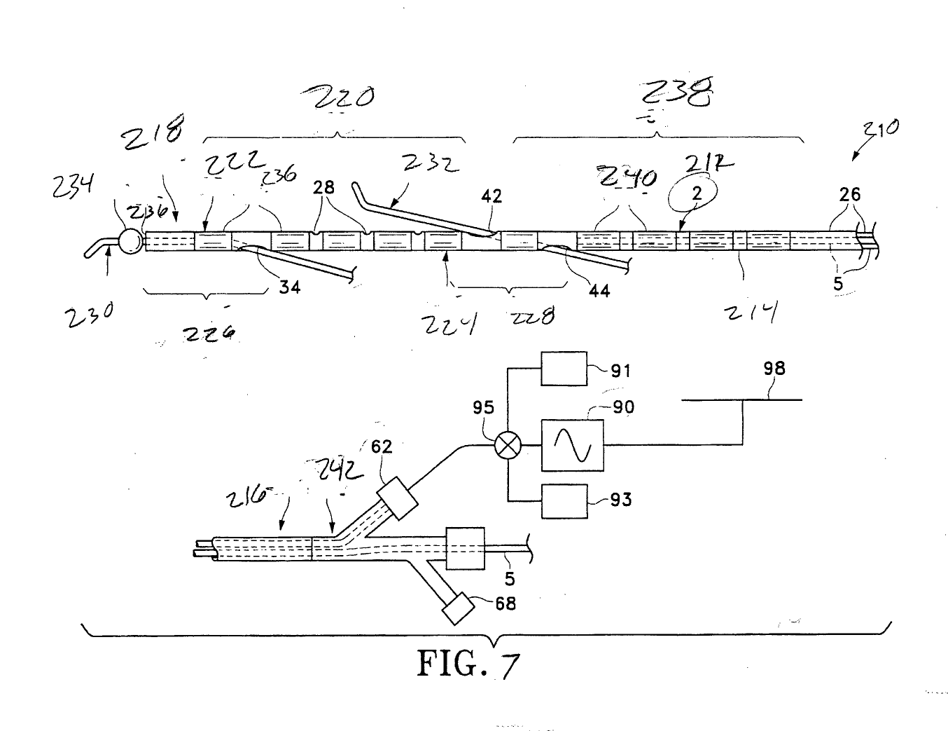

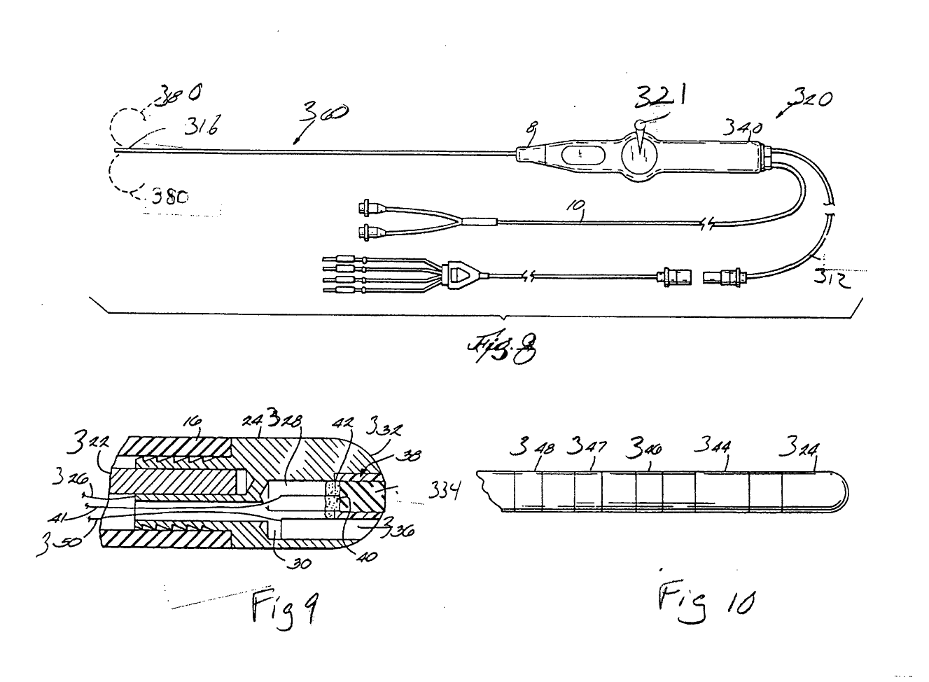

For typical applications where the thickness of pressure sensor die 120 is approximately 645 microns (micrometers), i.e., approximately 25 mils, and the total cavity height of the plastic housing 105 is approximately 135 mils, the nominal height of the protective dam 150 is in the range of 774-1,548 .micrometers, i.e., approximately 30-60 mils. Now referring to FIGS. 3 and 4, MEMS pressure sensor 101 with selective encapsulation 101 in accordance with another embodiment is depicted in which a vent cap 170 serves as a protectant. MEMS pressure sensor 101 includes vent cap 170 covering, sealing or otherwise encapsulating the wire bond cavity region instead using an encapsulation gel to fill the wire bond cavity. Vent cap 170 has a vent aperture 171 in the center which permits pressure sensor diaphragm 121 to receive unmolested ambient pressure. The prior art problem of gel over expansion is avoided by not having to fill the wire bond cavity with encapsulation gel.

Formation MEMS pressure sensor 101 employs similar steps as described for the formation of MEMS pressure sensor 100 including attaching pressure sensor die 120 to plastic housing 105 (partially shown), wire bonds 140 which electrically connect pressure sensor bond pads 122 to lead frame 130, and the construction of protective dam 150.

However, after protective dam 150 has been constructed on a top surface of pressure sensor die 120, vent cap 170 is placed over the device. The outer edges of vent cap 170 mate with plastic housing 105. The lower surface of the center portion of vent cap 170 is pressed down against protective dam 150. Sealing vent cap 170 takes place by curing the device at high temperature. Alternatively, an adhesive material can be used to seal vent cap 170. Also, various combinations of heat curing and adhesive may be employed to seal vent cap 170.

Preferably, vent cap 170 is formed from a plastic material which is compatible with plastic housing 105 and protective dam 150. In alternative embodiments, vent cap 170 may be constructed from metal. However, for a metal embodiment, adequate clearance must be provided between vent cap 170 and wire bonds 140 so as to preclude electrical shorting of wire bonds 140 to vent cap 170. The limitations of the height of protective dam 150 are similar to those described for MEMS sensor 100.

As shown in FIG. 3, vent cap 170 has an offset in the center portion where it contacts protective dam 150. The purpose of the offset is to optimize the height of the protective dam 150 with respect to wire bonds 140 and plastic housing 105. However, alternative embodiments may not need the offset.

Now referring to FIG. 5, a MEMS pressure sensor 102 with selective encapsulation in accordance with yet another embodiment of the present invention is depicted. In this embodiment, protective dam 150 is formed at the wafer level by bonding a cap wafer 151 to a device wafer 125 by means of a glass frit 152 or other suitable adhesive. A preliminary step in the fabrication of MEMS sensor 102 is to form a plurality of sensor devices on a substrate such as device wafer 125. FIG. 5 illustrates diaphragms 121 and wire bond pads 122 of a typical sensor device.

Independent of the sensor device formation on device wafer 125, a second wafer sometimes referred to as a cap wafer 151 is patterned with a plurality of diaphragm apertures 153, device channels 154 and cut lines 155. A subsequent step is to form a bonding area by depositing a glass frit pattern by screen printing or other means on cap wafer 151. Cap wafer 151 is then aligned and bonded to device wafer 125. The cap/device wafer combination is then heat cured and diced into individual pressure sensor dies 120 having a protective dam 150 attached.

FIG. 6 is a cross sectional view of encapsulated device 102 which further illustrates diaphragm aperture 153. Each of pressure sensor dies 120 is attached to housing 105 as described in previous embodiments. Wire bonding is similarly accomplished by connecting wire bonds 140 between wire bond pads 122 and lead frame 130. A wire bond cavity region is formed between the protective dam 150, i.e., the combination of portions cap wafer 151 and glass frit pattern 152, and housing 105. The wire bond cavity is filled with lo encapsulation gel 160 similar to the previously described embodiments. The limitations of the height of the protective dam 150 are similar to those described with respect to MEMS sensor 100.

FIG. 7 shows one embodiment of a tissue ablation device assembly 210 with an ablation catheter 212 which has an elongate body 214 with a proximal end portion 216 and a distal end portion 218. Distal end portion 218 is shown to include an ablation element 220 which is bordered on each of two ends 222 and 224 by distal and intermediate guidewire tracking members 226 and 228, respectively.

Anchors can be provided by the distal and intermediate guidewire tracking members 226 and 228. These guidewire tracking members are generally shown in FIG. 7 to be slideably engaged over distal and proximal guidewires 230 and 232, respectively, to form a “multi-rail” catheter system. Guidewire 230 is further shown to include a stop 234 that is radially enlarged with a diameter which is larger than the diameter of the first distal guidewire port 236. The stop 234 provides one positioning means for placing the distal guidewire tracking member 230 at a predetermined location along the guidewire to anchor it in that position in the anatomy. In addition to the use of the stop mechanism 234 shown, other structures may be employed to provide relative positioning of the catheter over the guidewire, such as by use of an expandable member on the guidewire to internally engage the guidewire tracking lumen, as would be apparent to one of ordinary skill.

In one embodiment, the ablation element 220 includes a plurality of electrodes 236 which are variously positioned along the length of the elongate body 214. A second ablation element 238 can be provided and in embodiment includes a second plurality of electrodes 240. The electrodes of these ablation elements are adapted via electrode leads to at least one ablation actuator and also to instruments that can be used to monitor intercardiac electrical signals and to artificially pace cardiac contractile rhythm via the electrodes. In the variation shown, a common bundle of electrode leads 240 couple the various electrodes to the proximal coupler 242.

FIG. 8 illustrates a catheter 320 having a handle 340 from which a flexible body 360 extends. Flexible body 360 extends from one end 380 of handle 340 while ultrasonic cable 310 and a combination electrode/thermistor cable 312 extend from the other end 314 of handle 340. Distal end 16 of flexible body 360 is steerable, as suggested by the dashed lines, in a conventional manner using a steering lever 320 mounted to handle 340. Lever 321 which controls one or more steering cables 322, as is conventional. Distal end 316 has an RF transmitting tip 324 secured thereto. Transmitting tip 324 is connected to an appropriate RF energy source, not shown, through lead 326 which extends along flexible body 360, through handle 340 and through combined cable 312.

Tip 324 has an axially extending bores 328 formed from its distal end 332. Bore 328 is used to house a sensor 336 which is a piezoelectric or pressure sensor. In one embodiment, illustrated in FIGS. 8-10, the ablation apparatus exemplified by the use of RF transmitting tip 324, also includes three ring electrodes 346, 347, 348 positioned in a proximal direction, that is towards handle 340 relative to tip electrode 324. Electrodes 346-348 (spaced 2.5 mm apart) are used to record electrical signals produced by the heart (electrocardiograms) for endocardial mapping using a multichannel EKG machine as is known in the art. Sensor 336 is coupled to combination cable 312 through a lead 350 extending from sensor 336, to flexible body 360, through handle 340 and into combination cable 312.

Sensor 336 is used to provide endpoint information regarding the end of the ablation. The RF ablation system, used to ablate the tissue can be of convention design such as that sold by EP Technologies, Inc. of Sunnyvale, Calif. In this embodiment, ablation and the end-point of ablation structures are in a single catheter which permits real time determination about the end-point of the ablation process. The amount or volume of tissue ablated can thus be constantly monitored during the procedure so that neither too little or too much tissue is ablated for maximum control and effectiveness. It will be appreciated that in this embodiment, instead of using RF energy to ablate the tissue, microwave radiation, laser energy, cryoblation or endocardial injection/infusion, for example, can also be used.

The use of catheter 320 proceeds generally as follows. Distal end 316 of body 360 is directed to the appropriate site using conventional techniques and steering lever 320. Visualization of the tissue to be ablated and localization of the tip 324 can be provided by an ultrasonic transducer 334, ultrasonic transponder 344, and associated leads and cables coupled to a conventional ultrasonic imaging console, not shown. When tip 324 is at the site of the tissue to be ablated, RF generator, not shown, coupled to combination cable 312, is activated to produce RF radiation at tip 324 to ablate the tissue. The ablation is monitored by sensor 336 to help ensure that the proper volume of tissue is ablated. When the proper volume of tissue is ablated, body 360 is removed from the patient. Instead of the use of catheter 320 including an RF transmitter tip 324, the catheter could use an ablation fluid infusion tip. Also, preparatory to the ablation sequence, the suspect area can be temporarily suppressed to deadened using catheter 360 using lidocaine or iced saline solution.

In one embodiment, the sensor die includes a transducer element, including but not limited to a capacitive diaphragm or membrane, that is sensitive to some ambient condition and which, for optimal performance, should be free of encapsulation gel material.

In one embodiment, the sensor is a piezoelectric sensor with two metal plates to sandwich a crystal and make a capacitor. External force cause a deformation of the crystal and results in a charge which is a function of the applied force. In its operating region, a greater force results in more surface charge. This charge results in a voltage V=Q_{f}/C, where Q_{f} is the charge resulting from a force f, and C is the capacitance of the device.

The piezoelectric crystals act as transducers which turn force, or mechanical stress into electrical charge which in turn can be converted into a voltage. Alternatively, if a voltage is applied to the plates, the resultant electric field causes the internal electric dipoles to re-align which cause a deformation of the material.

### EXAMPLE 1

When ablating surface tissue, the laser may be positioned normal to the tissue surface and a single horizontal sweep may be sufficient. However, for a larger area, the laser may be placed in a holder that is attached to a computer system programmed to scan a two dimensional pattern. This may be accomplished by a horizontal sweep and a vertical displacement to place the laser beam in position to complete another horizontal sweep. Providing computerized movement of the laser beam in two directions facilitates multiple pulsing regimes. For instance, the laser beam may ablate tissue at a single localized spot to the desired depth, or in the alternative, the pulsing beam may impinge on a new area of tissue every sequential pulse thereby scanning a larger area of tissue with minimal removal of tissue. This sequential scan may be repeated until the desired depth or removal of tissue is completed.

Additionally, the laser may be placed in a holder in such a way that pivoting of the laser allows for sweeping motion in at least a ninety degree arc in both the x and y direction. Also, the laser may be place on an angle to the surface thereby projecting the energy beam at a predetermined angle. A single piezoresistor sensor or an array of sensors is positioned at the ablation site and can fully or partially surround the ablation site. The piezoresistor sensor or array of sensors is tuned to a specific resonance frequency of the ablated tissue. This resonance frequency is different from the resonance frequency of the non-ablated tissue. When the ablation has reached the sensor array and thus the desired endpoint, the array of sensors produces an electrical signal.

### EXAMPLE 2

Another embodiment of the present invention provides for multiple locations of impingement by at least one electromagnetic energy source that deliver energy to the target locations either simultaneously or sequentially. A single laser with a split beam or multiple lasers may be used to impinge on multiple locations. Several points or locations of impingement will generate an interference response within the impinged and surrounding tissue. The beam profile of the laser may include any known configuration, including circular, rectangular, broad and gaussian shaped beams. Each beam geometric configuration provides unique advantages. For instance, gaussian laser beams, having a higher intensity in the center than at the edges of the beam, are able to penetrate one layer of tissue and show the transition of another layer very early in the ablation process. At the exact moment, the beam penetrates through the first layer a mixed signal is generated that indicates another layer of tissue has been penetrated. This can easily be analyzed because the amplitude of the signal for the first layer will be dampened due to the signal of the next layer of penetration which is visible on a time dependent plot.

A rectangular beam profile, typically emitted by laser diodes, provides uniform irradiance of the tissue at point of impingement. The rectangular beam is able to detect irregularities on the surface of impinged tissue and provides information concerning the different layers being ablated. Rectangular beams are especially useful when smoothening of an irregular surface is required. Specifically, a surface masking material, such as a viscous gel, may be spread on the surface to fill void and provide a layer comprising the masking material and the irregularites of the surface. The array of pressure transducer sensors is positioned at an ablation site, at a distance of a few mirometers to 10 mm or more, say 50 mm, and can fully or partially surround the ablation site. The array of pressure transducer sensors is tuned to a specific resonance frequency of the ablated tissue. This resonance frequency is different from the resonance frequency of the non-ablated tissue. When the ablation has reached the pressure transducer sensor array and thus the desired endpoint, the array of sensors produces an electrical signal.

### EXAMPLE 3

Broad beam lasers emit a broad beam of electromagnetic energy that is capable of impinging a comparatively large surface area of tissue. Advantageously, this broad coverage of the tissue allows for detecting emitted signals of generated acoustic waves at multiple locations within the tissue. Impinging radiation of a sufficient energy excites tissue molecules during absorption and/or interaction therein. After excitation, some of the molecules within the tissue return to the ground state by radiation-less processes. The thermal radiation array of sensors of the present invention is tuned to a specific resonance frequency of the ablated tissue. This resonance frequency is different from the resonance frequency of the non-ablated tissue. When the ablation has reached the thermal radiation sensor and thus the desired endpoint, the array of thermal radiation sensors produces an electrical signal.

### EXAMPLE 4

In one embodiment, an ablation catheter is advanced into a targeted region where an ablation occurs and a lesion formed. As non-limiting examples, the ablation can occur within the heart, liver, prostrate gland, eye, the brain and the like. The catheter can include a light emitter that provides broadband and/or laser light to the lesion site. The ablation element of the catheter is energized whereby a lesion is formed in the surrounding tissue. Light from the emitter is scattered by the lesion.

A MEMS array of sensors is tuned to a specific resonance frequency of the ablated tissue. This resonance frequency is different from the resonance frequency of the non-ablated tissue. When the ablation has reached the MEMS array of sensors and thus the desired endpoint. The MEMS array of sensors produces an electrical signal. The MEMS array of sensor converts resonance frequencies into an electrical signal that can be processed with a computer and displayed graphically to the end-user of the ablation device. During surgical ablation, the operator obtains information about the lesion as it is being formed or, alternatively, can detect lesions that have already been formed. The resonance frequencies changes due to ablation of tissue, allowing for an existing lesion to be located as the ablation catheter is advanced over tissue. Moreover, the depth of the lesion causes a corresponding change in the resonance frequency. The operator can use this information to increase or decrease the energy delivered to the site varying the depth of the lesion or terminating the ablation procedure. The distance of the MEMS array of sensors from the ablated tissue can be less than a millimeter away to 20 millimeter away.

### EXAMPLE 5

In this example, a surgical system is used to create ablation in the prostate to treat BPH. The surgical system includes a capacitance sensor or an array of capacitance sensors of the present invention. The surgical system includes an electromagnetic ablation source, an output assembly, a power assembly, a cooling assembly, a processor, and/or other components. In some implementations, the electromagnetic source outputs electromagnetic radiation at a predetermined output wavelength and a predetermined output power. Output assembly is configured to deliver the output electromagnetic radiation to soft tissue of the patient to ablate the soft tissue. For example, output assembly may include an optical fiber. In some instances, the optical fiber may include a side-firing tip. The output assembly may be configured to deliver the output electromagnetic radiation to prostate tissue of the patient to ablate the prostate tissue. The power assembly receives an input power from an external power source and may, at least in part, the drive source and/or other components of system with the input power. The cooling assembly may cool the source during operation. The processor controls operation of the system.

The electromagnetic energy source can include a generation assembly, an amplification assembly, a wavelength adjustment assembly, and/or other components or assemblies. The generation assembly generates electromagnetic radiation at a predetermined fundamental wavelength and a predetermined fundamental power. The amplification assembly can be optically coupled with the generation assembly to receive electromagnetic radiation from generation assembly and amplify the power of the received electromagnetic radiation to that the electromagnetic radiation that is output to ablate the soft tissue will be output at the predetermined output power. The wavelength adjustment assembly can be optically coupled to amplification assembly, and can adjust the wavelength of the received electromagnetic radiation to the predetermined output wavelength. In some instance, the output wavelength is shorter than the fundamental wavelength.

The processor can execute a generation module, an amplification module, a cooling/temperature module, a temperature tuning module, and/or other modules. The processor can be a single unit or include a plurality of processors operatively linked to each other, and that various ones of the linked processors can be physically located locally to each other, or may be remote from each other. The processor can include a processor integral with the other system components and a central processing unit of a host computer to control and/or read out data from the system.

The capacitance sensor or array of capacitance sensors of the present invention can be part of the generation module and receives resonance frequency feedback from the generation assembly. The capacitance sensor or array of capacitance sensors of the present invention can be used to tune one or more components of wavelength an adjustment module to an operating temperature. The tuning performed can enhance the performance of the wavelength adjustment assembly, protect one or more of the components of the wavelength adjustment assembly, and provide feedback to stop the delivery of electromagnetic energy when the resonance frequencies change.

Although the invention has been particularly shown and described with reference to a preferred embodiment thereof, it will be understood by those skilled in the art that changes in form and detail may be made therein without departing from the spirit and scope of the invention.

What is claimed is:

1. A method of tissue ablation during a tissue ablation procedure, comprising:
apply ablation energy by using a tissue ablation device to create an ablation at a tissue site; and
detecting an ablation endpoint at the tissue site by using an ablation endpoint device with one or more sensors that are positioned to monitor the ablation, the one or more sensors being selected from at least one of, a piezoelectric and a silicon MEMS sensor;
detecting the ablation endpoint; and
ceasing delivery of ablation energy to the tissue site.

2. The method of claim 1, wherein the tissue ablation device is an electromagnetic tissue ablation device.

3. The method of claim 1, wherein the one or more sensors resonate at a frequency equal to a resonance frequency of the ablated tissue.

4. The method of claim 3, wherein the resonance frequency is different from a frequency of non-ablated tissue.

5. The method of claim 1, wherein the one or more sensors is coupled to an external detection device.

6. The method of claim 5, wherein the external device indicates when the one or more sensors is excited to its resonance frequency.

7. The method of claim 6, wherein the external device is coupled to the one or more sensors by at least one of, cable and wireless.

8. The method of claim 3, wherein achieving a specific resonance frequency is used to determine the endpoint of the tissue ablation procedure.

9. The method of claim 1, wherein as the ablation procedure proceeds tissue radiating from the ablation device is effected by the ablation.

10. The method of claim 9, wherein the the procedure reaches tissue where the one or more sensors is positioned, the detector notifies the physician with an indication that the procedure should be discontinued.

11. The method of claim 1, In one embodiment, the endpoint detection device has an array of sensors that are mounted to or encapsulated in a biocompatible material.

12. The method of claim 11, wherein the array of sensors is positioned at the tissue site and is at least one of fully and partially surround the tissue site.

13. The method of claim 12, wherein the array of sensors is tuned to a specific resonance frequency of ablated tissue.

14. The method of claim 1, wherein the one or more sensors are piezoresistive sensors with substrates and two opposed surfaces.

15. The method of claim 14, wherein a dielectric insulated layer is on a first surface of a substrate.

16. The method of claim 15, wherein a doped semiconductor layer is on a top of the dielectric insulated layer.

17. The method of claim 16, wherein the doped semiconductor layer has a high resistivity.

18. The method of claim 17, wherein the doped semiconductor layer is annealed to one or more regions to lower resistivity of the semiconductor layer and defines therein one or more sensor gauges of the annealed semiconductor material.

19. The method of claim 18, wherein one or more electrical contacts are adjacent to the annealed semiconductor material and overlay at least a portion of the annealed semiconductor material.

20. A method of activity monitoring implantable defibrillators or pace makers, comprising:
positioning one or more sensors to monitor activity of an implantable defibrillator or pace maker; and
in response to the monitoring taking an action relative to the implantable defibrillator or pace maker.
